(19) United States
(12) Patent Application Publication
(10) Pub. No.: US 2008/0167710 A1
(43) Pub. Date: Jul. 10, 2008

(54) Medical Device Having Regions With Various Agents Dispersed Therein and a Method for Making the Same

(76) Inventors: Vipul Bhupendra Dave, Hillsborough, NJ (US); Chao Chen, Edison, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/620,227

(22) Filed: Jan. 5, 2007

(51) Int. Cl. A61F 2/06

(52) U.S. Cl. 623/1.34; 623/1.42; 623/1.46

(57) ABSTRACT

A device is constructed with additives incorporated into its structure, such as a material that increases visibility of the device, while still maintaining desired mechanical characteristics such as high radial stiffness, minimized recoil values, and improved flexibility. The device can assume a wide range of geometries that are adaptable to various loading conditions. In order to include performance-enhancing additives to the medical device without affecting mechanical performance, the additives are localized in discrete regions of a polymer structure from which a medical device will be formed. For example, a medical device can be prepared from a polymer form such as a tube containing radiopaque agent localized at its ends or in a desired pattern along the device. Agent is evenly distributed throughout the device such that, for example, elution of a therapeutic agent will be more precisely controlled.

### FIELD OF THE INVENTION

The present invention relates to methods for making polymeric medical devices, such as intraluminal polymeric drug-eluting stents. In particular, devices are formed from polymers blended with materials that are localized within regions of the polymeric device minimizing impact on the mechanical performance of the device.

### BACKGROUND OF THE INVENTION

Medical devices can be constructed from biodegradable materials such as polymers. For example, a stent constructed from a biodegradable polymer is a medical device that is implanted into a vessel. The stent exerts an acute and/or chronic outward force that will help to remodel a vessel to its intended luminal diameter. The stent may also contain a therapeutic agent that is delivered to the vessel at a desired location.

Forming a medical device from bioabsorbable polymers must be accomplished in such a manner as to insure that the device maintains patency when implanted into a vessel or other conduit within a body. For example, a polymeric stent is typically implanted into a vessel by expansion with a balloon or some other expandable means. It is crucial to ensure that the stent impinges upon the inner wall of the vessel. After expansion, however, the polymer stent will experience shrinkage or recoil that causes it to lose apposition. The performance of the polymeric device may be enhanced utilizing certain polymer blends and additives to achieve desired mechanical properties. The blends and additives help to prevent excessive radial recoil upon deployment, exhibit sufficient fatigue resistance and exhibit sufficient ductility so as to provide adequate coverage over the full range of intended expansion diameters.

Maximizing the performance of the polymeric medical device also requires that it be accurately placed. For example, a drug-eluting polymeric stent must be placed within a vessel at the diseased site. If the stent is mis-placed, the diseased site will not be properly treated and will require the implantation of a new stent. Placement of a new stent, however, could be complicated by the presence of the previously misplaced stent. Thus, it is desired for at least part of the device to be visible from outside the patient to ensure proper placement. X-rays are one monitoring means employed to determine the position of a medical device as it is being implanted within a patient. Radiopaque additives, which are visible by X-Ray, can be dispersed throughout a polymeric device.

In order to achieve adequate radiopacity as much as 20%-30% by weight of radiopaque agent may be required. The presence of the radiopaque agent dispersed throughout the tube can affect the mechanical properties of the polymeric device such as making the device too brittle. Moreover, incorporating radiopaque agent directly within the polymeric device complicates the manufacturing process. One alternative to incorporating the radiopaque agent directly into the polymer(s) used to construct the device is to place maker bands directly on the device at a desired location. The drawback to this approach, however, is that radiopaque agents tend to be metallic and do not easily bond onto polymeric structures. Thus, the link between the radiopaque agent and device will be purely mechanical and may not exhibit sufficient strength to withstand implantation.

Medical devices may contain a therapeutic agent that further ensures proper modeling of a conduit, such as a vessel, by preventing restenosis or neointimal hyperplasia. Polymeric devices improve the delivery of the therapeutic drug and are formed such that the drug is dispersed within the polymer matrix. In order to place the drug within the polymer matrix a solvent may be employed. The removal of the solvent causes the polymer to assume a structure that can adversely affect the mechanical performance of the device. One solution is to coat the device with the drug such that the polymer is not affected.

A coated device, however, does not provide the same level of therapeutic agent elution as a device having drug dispersed within the polymer matrix. Typically, the bulk quantity of the therapeutic agent resides in the abluminal surface of the coating and is not dispersed there through. Even if the drug is dispersed throughout the coating, the coating layer is, by nature, a thin layer that provides a lower capacity to carry therapeutic agents. Finally, it is difficult to localize coating to a discrete area on the medical device in a manner that will ensure uniform dosage of therapeutic agent.

Currently, there is no polymeric medical device or method for making a polymeric medical device wherein the mechanical performance is not adversely affected by the addition to the medical device of materials that enhance visibility and/or carry therapeutic agents.

### SUMMARY OF THE INVENTION

A medical device is constructed from a polymeric composition having properties such as increased visibility of the device and the ability to deliver therapeutic and other agents. The device is constructed with additives incorporated into the polymeric structure, such as a material that increases visibility of the device, while still maintaining desired mechanical characteristics such as high radial stiffness, minimized recoil values, and improved flexibility. The device can assume a wide range of geometries that are adaptable to various loading conditions and may be utilized for any number of medical applications including vessel patency devices, such as vascular stents, biliary stents, ureter stents, vessel occlusion devices such as atrial septal and ventricular septal occluders, patent foramen ovale occluders and orthopedic devices such as fixation devices.

In order to include performance-enhancing additives in the medical device without affecting mechanical performance, the additives are localized and arrayed in discrete regions of a polymer structure from which a medical device will be formed. For example, a medical device can be prepared from a polymer form such as a tube containing radiopaque agent localized at its ends. The polymer tubes have discrete transition regions demarcating the radiopaque agent from the regions not containing the agent but do not exhibit dimensional discontinuity. The method for forming the polymer tubes produces different compositions in different segments of the tube including varying the loading of a therapeutic agent along the length of the device formed from the tube.

More particularly, polymer structures with and without an agent are formed, sized, and then assembled by bonding appropriate segments. The bonding process employs a bonding or gluing agent. Other bonding or sealing processes may be employed such as localized heating and solvent vapor bonding. The localized heating process is performed within a short time period and in a small local area to minimize detrimental effects on therapeutic agents if present. Alternatively, an agent can be placed on a mandrel or other substrate. A polymer form advanced onto the substrate in such a way as to bring a discrete region of the form into contact with the agent. The agent is compressed between a stop and the form. After a length of time, the agent bonds with the polymer. Other approaches include laying down a coating on a mandrel or substrate using a nozzle, advancing a polymer form along the longitudinal length of the mandrel, toward the nozzle, and removing the nozzle when it comes into contact with the polymer form.

Tubes with and without an agent are prepared using either melt extrusion or solvent casting. The tubes are cut into desired dimensions depending upon the device to be formed there from. The tubes are then placed on a mandrel to form the localized regions. The order of placing the tube onto the mandrel or the manner in which it is cut is determined by the desired region in which the agent will be localized. For example, if it is desired to have a radiopaque agent at an end of a device, the tube containing the agent is placed on the mandrel first followed by the tube without the agent. Alternatively, if it is desired to have the radiopaque marker confined to a region along the longitudinal length of the device, then a portion of the tube without the agent can removed and replaced with a section of the tube containing the agent. Other patterns and designs for the localized regions can be formed in a similar manner as will be appreciated by one of skill in the art.

Once the tubes or sections of the tubes are aligned on the mandrel, they are bonded together. The tube is then formed into a medical device by a process such as laser cutting. Alternatively, the tubes with and without agent can be formed into medical devices. The completed devices can then be cut or sized so that discrete sections of the device with and without the agent can be bonded together.

An apparatus for carrying out the steps of making the medical device includes a mandrel for mounting the tubes thereon, a dispensing device for providing bonding agent, a gripping device for holding polymer forms onto the mandrel and pressing them together, and a controller which allows for manual and automatic actuation of the process for joining the polymer forms together. As described above, at least two polymer forms are mounted onto the mandrel. The mandrel is mounted onto a block such that it can be rotated or moved longitudinally. This allows for bonding agents to be applied to a location on the mandrel where the at least two tubes will be joined. After the bonding agent is applied the tubes are brought into tight contact.

The bonding solution is deposited onto the mandrel using the dispensing device. The device is extended into position using a guide such as a pneumatic cylinder. The dispensing device can be adjusted with varying degrees of freedom to achieve proper location over the mandrel where solution will be deposited. The amount of bonding solution dispensed depends on a dispense controller which governs the amount of bonding agent dispensed. After bonding agent is placed on the mandrel, the gripping device is actuated to pull together the tube sections on the mandrel. In one embodiment, the gripping device comprises fingers connected to weights through a pulley system. The load is applied to the polymer forms when a pneumatic cylinder is retracted allowing the fingers to be pulled together on a linear guide. Fittings located on the mandrel engage the fingers to clamp the tubes sections together.

A wiping assembly removes excess bonding agent from the polymer forms. The wiping assembly comprises several rollers through which a wiping surface such as a film is pulled. The film is dispensed from one roller through an idle roller and wound onto another driven roller. As the film is pulled over the rollers, a pneumatic cylinder lifts the front roller so that the film is pushed into contact with the mandrel and absorbs any excess bonding solution. The film contacts as it is pulled through the rollers wiping off the excess bonding solution.

### BRIEF DESCRIPTION OF THE DRAWINGS

The foregoing and other features and advantages of the invention will be apparent from the following, more particular description of preferred embodiments of the invention, as illustrated in the accompanying drawings.

FIG. 1A is a side view of tube constructed from at least on polymeric material in accordance with the present invention.

FIG. 1B is side view of a tube constructed from at least one polymeric material having at least one additive in accordance with the present invention.

FIG. 1C is a side view of a tube constructed from at least one polymeric material having at least one additive showing the top of a finite element defined thereon.

FIG. 1D is an orthogonal view of the finite element shown in FIG. 1C.

FIG. 2 is a side view of a tube constructed from the tubes having the materials of FIGS. 1A and 1B.

FIG. 3 is a side view of a tube constructed from the tubes having the materials of FIGS. 1A and 1B.

FIG. 4 is a planar view of a medical device constructed from the tube of FIG. 2.

FIG. 4A is a planar view showing the medical device of FIG. 4 in greater detail.

FIG. 5 is a planar view of a section of a medical device constructed from the tube of FIG. 3.

FIG. 6 is a front view of an apparatus for constructing a tube in accordance with the present invention.

FIG. 7 is a planar view of the apparatus of FIG. 6.

### DETAILED DESCRIPTION OF THE INVENTION

Implantable medical devices may be fabricated from any number of suitable biocompatible materials, including materials such as polymeric materials. The internal structure of these polymeric materials may be altered utilizing mechanical and/or chemical manipulation. These modifications may be utilized to create devices having specific characteristics such as crystalline and amorphous morphology and orientation.

In accordance with the present invention, implantable medical devices may be fabricated from any number of biocompatible polymeric materials. These polymeric materials may be non-degradable, biodegradable and/or bioabsorbable. These polymeric materials may be formed from single polymers, blends of polymers and blends of polymers and plasticizers. In addition, other agents such as drugs and/or radiopaque agents may be blended with the polymeric materials or affixed or otherwise added thereto. A number of chemical and/or physical processes may be utilized to alter the chemical and physical properties of the materials and ultimately the final devices.

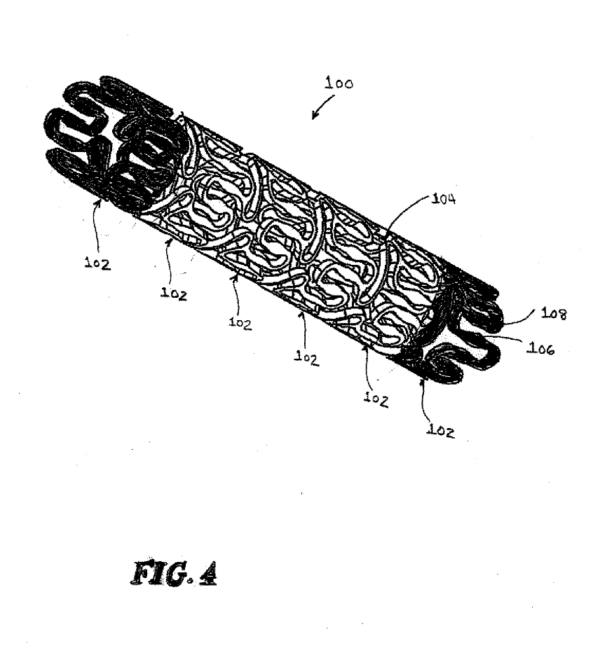

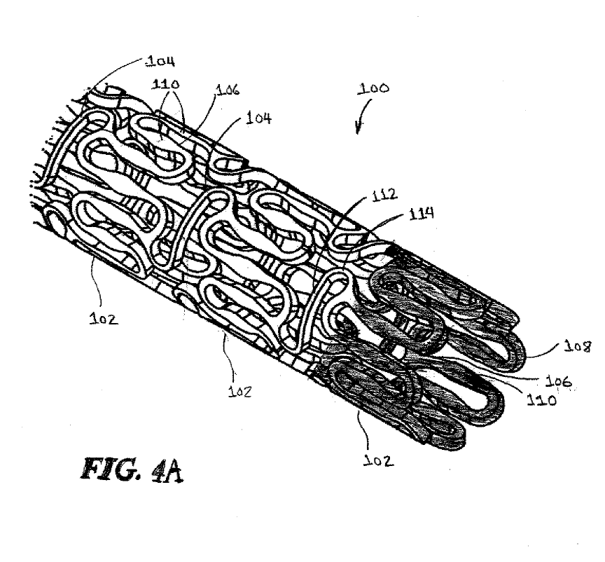

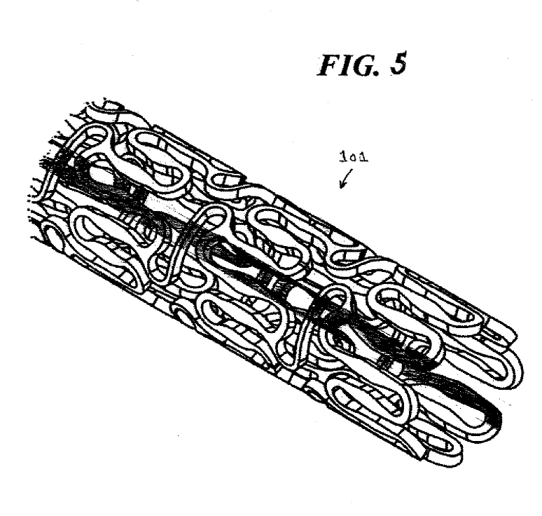

One example of a medical device that can be manufactured from the materials described above is a stent. A stent is commonly used as a generally, but not necessarily, tubular structure left inside the lumen of a duct to relieve an obstruction. Referring to FIGS. 4-5, there is illustrated a stent 100 that is manufactured in accordance with the present invention. As shown in FIG. 4, the stent 100 comprises a plurality of hoop components 102 interconnected by a plurality of flexible connectors 104. The hoop components 102 are formed as a continuous series of substantially longitudinally or axially oriented radial strut members 106 and alternating substantially circumferentially oriented radial arc members 108. Although shown in planar view, the hoop components 102 are essentially ring members that are linked together by the flexible connectors 104 to form a substantially tubular stent structure. The combination of radial strut members 106 and alternating radial arc members 108 form a generally sinusoidal pattern but other patterns may be formed such as a zig-zag pattern by connecting radial strut members directly together. Although the hoop components 102 may be designed with any number of features and assume any number of configurations, in the exemplary embodiment, the radial strut members 106 are wider in their central regions 110. This design feature may be utilized for a number of purposes, including, increased surface area that assists in delivering a therapeutic agent, as discussed in greater detail below.

As shown in FIG. 4A, the flexible connectors 104 are formed from a continuous series of flexible strut members 112 and alternating flexible arc members 114. The flexible connectors 104, as described above, connect adjacent hoop components 102 together. In this exemplary embodiment, the flexible connectors 104 have a substantially N-shape with one end being connected to a radial arc member on one hoop component and the other end being connected to a radial arc member on an adjacent hoop component. As with the hoop components 102, the flexible connectors 104 may comprise any number of design features and any number of configurations. In the exemplary embodiment, the ends of the flexible connectors 104 are connected to different portions of the radial arc members of adjacent hoop components for ease of nesting during crimping of the stent. It is interesting to note that with this exemplary configuration, the radial arcs on adjacent hoop components are slightly out of phase, while the radial arcs on every other hoop component are substantially in phase. In addition, it is important to note that not every radial arc on each hoop component need be connected to every radial arc on the adjacent hoop component.

A wide variety of designs may be utilized for the flexible connectors or connectors in an intraluminal scaffold or stent. For example, in the design described above, the connector comprises two elements, substantially longitudinally oriented strut members and flexible arc members. In alternate designs, however, the connectors may comprise only a substantially longitudinally oriented strut member and no flexible arc member or a flexible arc connector and no substantially longitudinally oriented strut member.

The generally tubular structure of the stent 100 provides either temporary or permanent scaffolding for maintaining patency of body conduits, such as arteries. The stent 100 is usually inserted into the lumen of a body conduit in a non-expanded form and are then expanded autonomously (or with the aid of a second device) in situ. When used in coronary artery procedures for relieving stenosis, stents are placed percutaneously through an artery such as the femoral artery. In this type of procedure, the stent 100 is delivered on a catheter and are either self-expanding or, in the majority of cases, expanded by a balloon.

It should be understood that the present invention may be utilized not only in connection with an expandable intraluminal vascular graft for expanding partially occluded segments of a blood vessel, duct or body passageways, such as within an organ, but may so be utilized for many other purposes as an expandable prosthesis for many other types of body passageways. For example, expandable prostheses may also be used for such purposes as: (1) supportive graft placement within blocked arteries opened by transluminal recanalization, but which are likely to collapse in the absence of internal support; (2) similar use following catheter passage through mediastinal and other veins occluded by inoperable cancers; (3) reinforcement of catheter created intrahepatic communications between portal and hepatic veins in patients suffering from portal hypertension; (4) supportive graft placement of narrowing of the esophagus, the intestine, the ureters, the uretha, etc.; (5) intraluminally bypassing a defect such as an aneurysm or blockage within a vessel or organ; and (6) supportive graft reinforcement of reopened and previously obstructed bile ducts. Accordingly, use of the term “prosthesis” encompasses the foregoing usages within various types of body passageways, and the use of the term “intraluminal graft” encompasses use for expanding the lumen of a body passageway. Further in this regard, the term “body passageway” encompasses any lumen or duct within the human body, such as those previously described, as well as any vein, artery, or blood vessel within the human vascular system.

The stent 100 is employed by first identifying a location where the stent 100 will be deployed, for example, a site within the vessel in a patient's body where a stenotic lesion or vulnerable plaque is located. Upon identifying the desired deployment location, a delivery device, such as a catheter carrying the stent 100 crimped to a distal end of the catheter such that the stent 100 is in its closed configuration, is inserted within the vessel in the patient's body. The catheter is used to traverse the vessel until reaching the desired location wherein the distal end of the catheter is positioned at the desired location, for instance the lesion, within the vessel. At this point, the stent 100 is deployed to its open configuration.

The stent 100 can be deployed via balloon expansion, self-expansion, or a balloon assist self-expansion delivery system. Typically, a self-expanding stent is constructed from a super-elastic shape memory material. The self-expanding stent constrained until delivered to the desired site. The constraint is then removed allowing the stent to expand to its remembered shape. An expandable stent is crimped to an expansion member, such as a balloon whereby the balloon is inflated to expand and deploy the stent. The benefit of using a combination system is that the stent 100 will not be crimped to lower profiles and upon deployment will self-expand to a certain value and can be further expanded to the desired dimension by balloon expansion. After the stent is expanded the inflatable member is collapsed (in the case of a balloon expandable stent) and the catheter is removed from the deployment site and the patient's body.

Once the stent is in place, the conduit should assume a shape that ensures the proper flow of fluids there through. Nonetheless, additional procedures may be required at other locations downstream from the location where the stent has been placed. In performing these procedures, the presence of pre-placed stents must be taken into account as the stent must be passed through to reach the downstream site. Thus, it is advantageous to manufacture a stent from a biodegradable substance, such as a polymer.

In general, the stent 100 can be constructed from biodegradable or bioabsorbable polymer compositions. The type of polymers used can degrade via different mechanisms such as bulk or surface erosion. Bulk erodible polymers include aliphatic polyesters such poly (lactic acid); poly (glycolic acid); poly (caprolactone); poly (p-dioxanone) and poly (trimethylene carbonate); and their copolymers and blends. Other polymers can include amino acid derived polymers; phosphorous containing polymers [e.g., poly (phosphoesters)] and poly (ester amide). Surface erodible polymers include polyanhydrides and polyorthoesters. The stent 100 can be made from combinations of bulk and surface erodible polymers to control the degradation mechanism of the stent. For example, the regions that are under high stress can be made from a polymer that will retain strength for longer periods of time, as these will degrade earlier than other regions with low stress. The selection of the polymers will determine the absorption of stents 100 that can be very short (few weeks) and long (weeks to months).

The stent 100 is generally fabricated from forms, such as tubes, made from the polymers, blends, additives and agents described above. The forms are processed, for example, by laser cutting to form the stent 100. A form such as a tube used to prepare bioabsorbable stents 100 can be fabricated either by melt or solvent processing. The preferred method will be solvent processing, especially for the stents that will contain drug. These tubes can be converted to the desired design by excimer laser processing. Other methods to fabricate the tubes from which the stent is crafted can be injection molding using supercritical fluids such as carbon dioxide.

Different processes may provide different structures, geometries or configurations to the bioabsorbable polymer being processed. For example, tubes processed from rigid polymers tend to be very stiff, but may be very flexible when processed via electrostatic processing or lyophilization. In the former case, the tubes are solid, whereas in the latter case, the tubes are porous. Other processes provide additional geometries and structures that may include fibers, microfibers, thin and thick films, discs, foams, microspheres and even more intricate geometries or configurations. Melt or solution spun fibers, films and tubes may be further processed into different designs such as tubular, slide and lock, helical or otherwise by braiding and/or laser cutting. The differences in structures, geometries or configurations provided by the different processes are useful for preparing different drug delivery devices with desired dimensions, strengths, drug delivery and visualization characteristics. The fibers, films or tubes may be laser cut to a desired geometry or configuration such as in the shape of a stent. Other machining techniques may also be utilized.

The bioabsorbable compositions to prepare the stent 100 may also include agents that enhance the performance of the stent 100 such as biologically active materials, therapeutic drugs, radiopaque materials, and other materials, devices or the like. Typically, various drugs or agents are incorporated into the medical device by a affixing it to the surface of the device via a polymer coating that contains the drug within its matrix. In particular, the coating is comprised of bioabsorbable polymers into which the drugs or other agents are incorporated. Shortcomings are exhibited in the drug elution characteristic of coated devices. For example, the bulk quantity of the therapeutic agent resides in the abluminal surface of the coating and is not dispersed evenly there through. Even if the drug is dispersed throughout the coating, the coating layer is, by nature, a thin layer that provides a lower capacity to carry therapeutic agents An alternative is to incorporate the therapeutic agents into the matrix of bioabsorbable materials comprising the device. The drugs or agents incorporated into the matrix of bioabsorbable polymers will be in an amount that can be the same as, but typically greater than, the amount of drugs or agents provided in a coating. This is accomplished by forming a solution of a polymer and agent(s) and converting the solution into a tubular structure. For example, a homogenously mixed solution containing 1-40% agent dissolved in 15-18% polymer via a solvent is deposited on a mandrel by a solvent casting process. In particular, a solution having 15% polymer; 8% radiopaque agent; 4% therapeutic agent; and 73% solvent may be used. After depositing the solution on a mandrel, the solvent is removed leaving the agent dispersed uniformly in the polymeric structure. This results in a polymeric structure having 55% polymer, 15% therapeutic agent and 30% radiopaque agent. As described in greater detail below, the polymer may comprise a combination of polymers and include additives and plasticizers. Alternatively, coating and incorporating the agent into the polymer matrix can be combined.

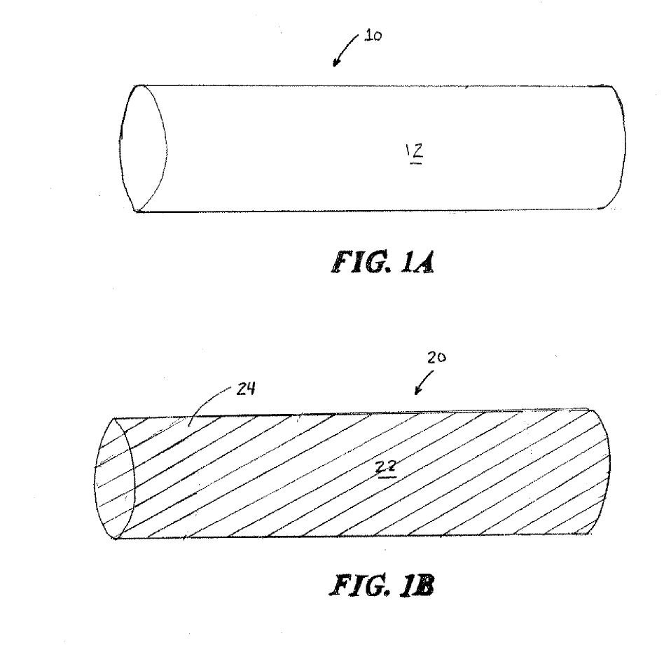

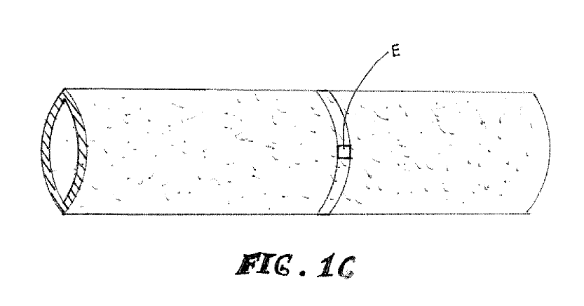

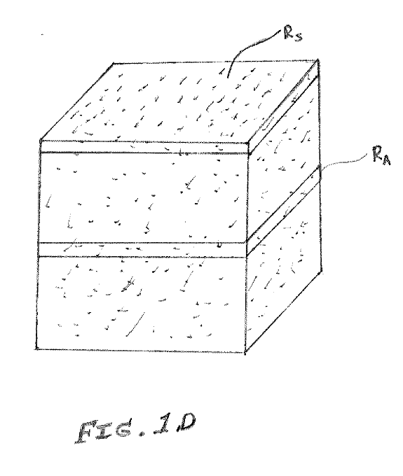

FIG. 1C shows a polymer tube 21 having a therapeutic agent in its polymer matrix. A device such as stent 100 can be constructed from tube 21. A finite element E having a total amount of agent T_{E} contained therein is located on tube 21. Finite element E exhibits the same properties as any other finite element of the same volume regardless of its location on tube 21, unless, of course, the properties of the tube vary along its length, i.e. a thicker region of the tube or varying material density due to differing agents, polymers, additives or differing morphology. Nonetheless, any finite element in a region of the tube having uniform properties will exhibit the same properties.

As shown in greater detail in FIG. 1D, the element E has a surface layer R_{S} of a finite thickness and a layer R_{A}, which can be located anywhere within the element E, excluding the surface layer R_{S} and having the same thickness as R_{S}. The total amount of agent in layers R_{S} and R_{A} may be defined as being T_{S} and T_{A} respectively. Assuming layers R_{S} and R_{A} have equal volumes V_{S} and V_{A} respectively, then in accordance with the principles of the present invention the tube 21 is constructed such that the agent is distributed within element E whereby T_{S}/V_{S} is substantially the same as T_{A}/V_{A}. In an alternate embodiment of the invention, the agent is distributed within element E such that greater than 10% of the total amount of agent is contained in the layer(s) other than the surface layer. Typical surface layer thicknesses vary from between five to fifteen microns.

Other agents can be added to the stent 100 such as radiopaque additives. These can include barium sulfate and bismuth subcarbonate and the amount can be from 5 to 30 percent as an example. Other radiopaque materials include gold particles and iodine compounds. The particle size of these radiopaque materials can vary from nanometers to microns. The benefit of small particle size is to avoid any reduction in the mechanical properties and to improve the toughness values of the devices. Upon polymer absorption, small particles will also not have any adverse effects on surrounding tissues.

Although it is desirable to have therapeutic and radiopaque agents within the polymer, the presence of these agents can adversely affect the mechanical properties of the polymeric device 100. Typically, polymer medical devices are constructed by cutting a desired device from a tube of polymeric material. As discussed above, the polymeric materials used to create the tube can be formed with various agents and additives mixed with the polymer. In accordance with the present invention, the agents or additives are localized in discrete regions of a polymer structure, such as a tube, from which the medical device 100 will be formed in order to minimize the impact on mechanical performance and enhance other performance features of the device 100 such as visibility.

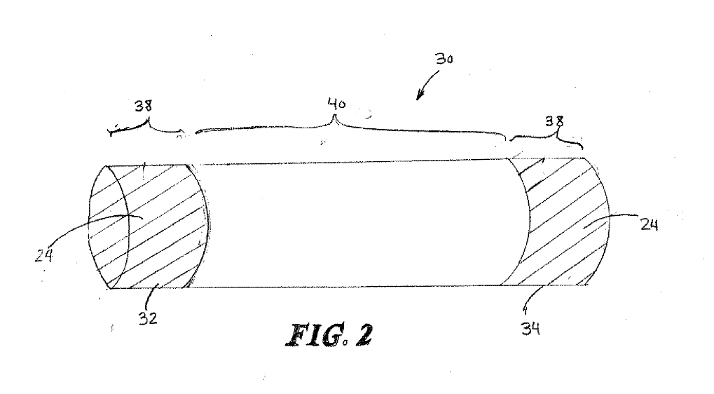

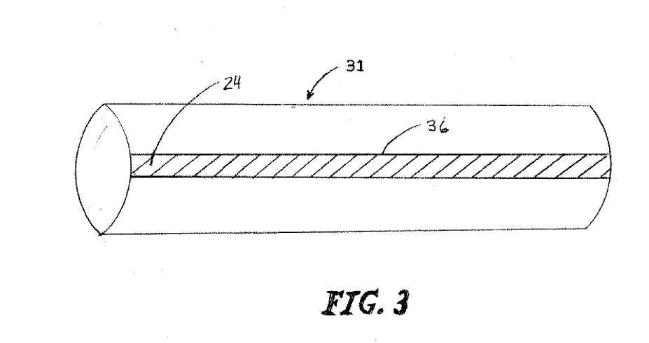

FIG. 1A shows a tube 10 constructed from a polymeric material or a blend of polymeric materials. Tube 10 has a body 12 that can be formed by extrusion or solution processing. A tube can be formed from a polymeric material with an additive and/or agent therein as described in detail below. For example, as seen in FIG. 1B a tube is constructed from a polymer with a radiopaque agent 24 within the body 22 of tube 20. Alternatively, tube 20 may have a therapeutic agent, not shown in the Figures. As shown in FIG. 2, tubes 10 and 20 can be combined such that a unitary tube 30 is produced wherein the polymer containing agent 24 is confined to the distal 32 and proximal 34 regions of the tube 30. The present invention contemplates a wide arrangement of regions of tube 30 where a polymer having agent 24 can be confined or located. For example, agent 24 may be confined to a single point or to a region forming a zig-zag pattern along the length of tube 30. As shown in FIG. 3, a tube 31 may be formed such that agent 24 is confined to a region 36 along the longitudinal length of tube 31. The tubes 30 and 31 have discrete transition regions demarcating the agent 24 from the regions not containing the agent but do not exhibit dimensional discontinuity. If the tubes 30 and 31 also contain a therapeutic agent, the loading of the therapeutic agent may be varied.

In accordance with the present invention tubes having discrete regions are generally formed from polymer structures such as tubes 10 and 20 wherein one tube 10 is formed without an agent and one tube 10 is formed with an agent 24. The tubes 10 and 20 are formed, sized, and then assembled by bonding appropriate segments. The bonding process employs a bonding or gluing agent. Other bonding or sealing processes may be employed such as localized heating and solvent vapor bonding. The localized heating process is performed within a short time period and in a small local area to minimize detrimental effects on therapeutic agents if present. Alternatively, an agent 24 can be placed on a mandrel or other substrate, not shown in the Figures. A polymer tube 10 is advanced onto the substrate in such a way as to bring a discrete region of the tube 10 into contact with the agent 24. The agent 24 is compressed between a stop and the form. After a length of time, the agent 24 bonds with the polymer of tube 10. Other approaches include laying down a coating on a mandrel or substrate using a nozzle, advancing a polymer form such as a tube along the longitudinal length of the mandrel, toward the nozzle, and removing the nozzle when it comes into contact with the polymer form.

It is important to note that it may be desirable to have an agent present throughout the entire tube from which the medical device will be formed, but in different quantities. Alternatively, it may be desirable to have a plurality of discrete regions each having a different agent contained therein. This may be accomplished by forming any number of tubes, depending on the discrete regions to be formed, each having one or more agents contained therein or differing amounts of agent contained therein. Each of the tubes are sized and bonded together to form the tube from which the medical device will be constructed. For purposes of illustration only, the present invention is described with reference to two tubes 10 and 20.

An example of a process employed to construct tube 30 and device 100 there from includes preparing tubes 10 and 20 using either melt extrusion or solvent casting process. Tube 20, containing agent 24, was prepared from PLGA with 10% citrate ester and 30% barium sulfate and tube 10 was prepared from PLGA with 10% citrate ester without barium sulfate from a solvent cast process. Tubes 10 or 20 could also be fabricated to contain therapeutic agents. The tubes 10 and 20 were dried at low drying temperatures (for example, ambient temperature up to 60° C.) to obtain a solvent level of about 20%. Solvent remains in the tubes 10 and 20 as completely dried tubes may not form adequate adhesion during the bonding process. Tubes 10 and 20 are cut to desired size (for example, 16 mm tube segment 40 cut from tube 10 without agent 24; and at least one 2 mm tube segment 38 cut from the tube 20 with agent 24). Alternatively, the tubes 10 and 20 and any additional tubes needed can be formed to have a desired length eliminating the need to form segments.

The segments 38 and 40 are placed on a mandrel in the following order: segment 38 with agent 24 followed by segment 40 without agent 24 (16 mm) optionally followed by another segment 38. Segments 38 (if multiple segments 38 are employed) need not be the same size or even shape and are shown as such for ease of illustrating an aspect of the present invention. A small gap of about 1 mm is left between the segments 38 and 40. These gaps are filled with a bonding medium using a dilute PLGA solution with about 4 to 8% polymer concentration. The bonding medium may also include agents such as therapeutic agents or radiopaque materials and be constructed from the same material as either tube segments 38 or 40 are constructed from.

The segments 38 and 40 are brought into contact and excess solution squeezed from the gaps is removed. The segments 38 and 40 are allowed to dry in a nitrogen atmosphere at ambient temperature, and were then dried at 60° C. or at a suitable temperature to remove remaining solvent to prepare a good bond between the tube and the marker bands. Tube 30 can be prepared in this manner with the proximal and distal ends containing agent 24, such as 30% barium sulfate. Alternatively, a tube such as 31 can be prepared by removing a longitudinal segment from tube 10 and replacing it with a segment from tube 20 that has been sized to fit where the segment was removed from tube 10. Other patterns and designs for the localized regions can be formed in a similar manner as will be appreciated by one of skill in the art. As shown in FIGS. 4 and 4A a medical device such as a stent 100 can be cut or etched from tube 30, for example, by a laser cutting process or acid etching process. Agent 24 is localized to the ends of stent 100. Similarly, a stent 101, a segment of which is shown in FIG. 5, was prepared from tube 31 and has agent 24 localized to a region along the longitudinal length of the stent 101.

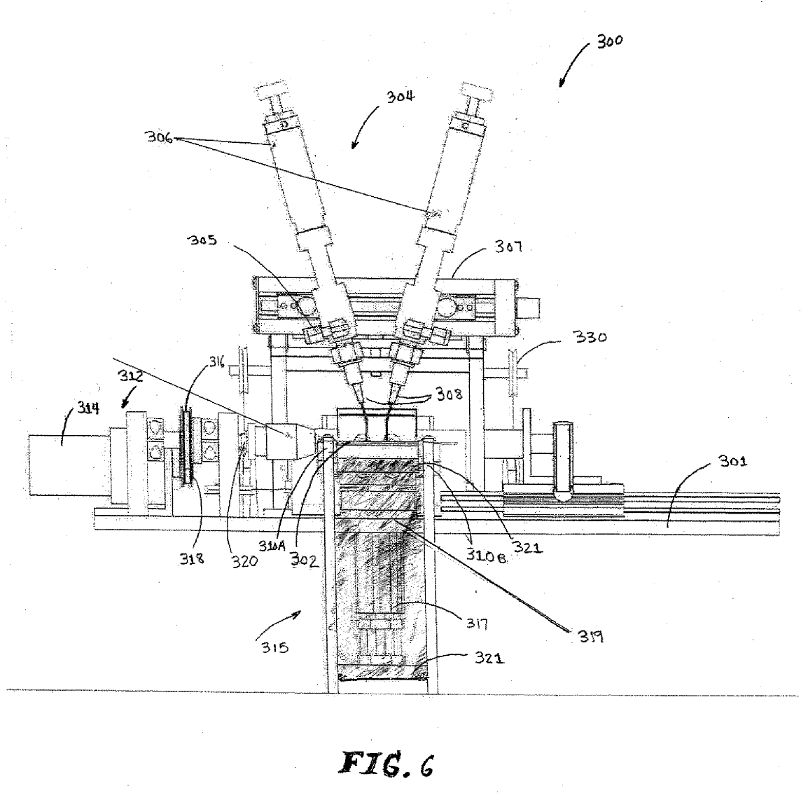

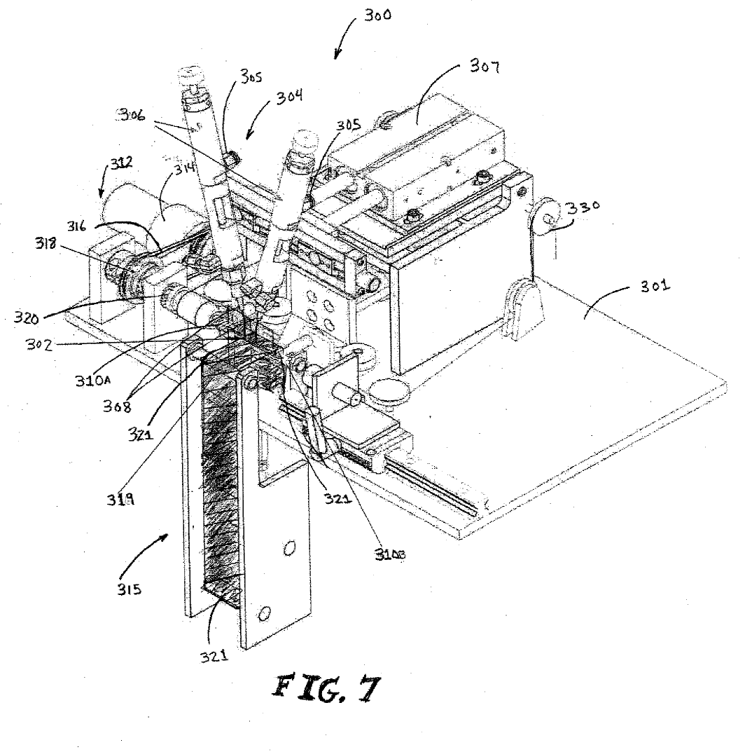

FIGS. 6 and 7 show an apparatus 300 that prepares the tubes with agent 24 localized to a desired region. The apparatus 300 is mounted on a base 301 and includes a mandrel or substrate 302 for mounting the tube segments 38, 40 thereon, a bonding agent dispenser 304, a gripping device 310 for holding polymer forms such as tube segments 38 and 40 onto the mandrel or substrate 302 and pressing them together, and a controller, not shown in the drawings which allows for manual and automatic actuation of the process for joining the segments together. Mandrel 302 is rotated by a drive mechanism 312 mounted onto base 301. The drive mechanism 312 may comprise a variable speed motor 314 connected to a belt 316 and pulley 318 that is mounted onto drive shaft 320. The drive mechanism 312 can be mounted such that it can be translated longitudinally. For example, the drive mechanism may be pushed in a direction towards mandrel 302 thereby translating mandrel 302 longitudinally. Alternatively, mandrel 302 can be mounted to slide along drive shaft 320. This allows for bonding agents to be applied to a location on the mandrel where the at least two segments 38 and 40 will be joined. Preferably, bonding solution-dispensing system 304, described below, can be translated along mandrel 302 when applying bonding agents.

Bonding solution-dispensing system 304 deposits the bonding solution onto the mandrel using at least one, but preferably two dispensers 306. The tips or heads 308 of the dispensers 306 are extended into position using guided pneumatic cylinder 307. The tips or heads 308 can be adjusted forward/backward, up/down and together/apart using adjusting screws to achieve proper location over the mandrel 302. In one embodiment of the present invention, dispenser 306 comprises a pneumatic syringe. Alternatively, the bonding agent may be dispensed manually from a container. A fitting 305 allows for a conduit to be connected that links dispenser 306 to a supply source for bonding solution.

Clamping fingers 310A and 310B pull together the segments 38 and 40 onto the mandrel 302 after the bonding solution has been deposited. The operator can actuate a crank 330 connected to the fingers through a pulley system. Alternatively, a pneumatic drive connected to fingers 310A and 310B can be employed to pull the segments together in the desired direction. For example, the load can be applied to the segments 38 and 40 when a pneumatic cylinder is retracted allowing the fingers to be pulled together on a linear guide. Fittings, not shown in the drawings, are placed onto the mandrel 302 prior to mounting on the apparatus 300 to engage the fingers 310A and 310B. This aids in clamping the tube segments 38 and 40 together after deposition of the bonding solution.

After the segments 38 and 40 are pressed together excess bonding agent will be present on the joined together segments. A wiping assembly 315 removes the excess bonding agent. The assembly 315 comprises a wiping film 319, such as low-density polyethylene film, that is mounted on rollers 321. For example, the film 319 wraps from one roller through the idle rollers and is wound onto another driven roller. As the film 321 is pulled over the rollers, a pneumatic cylinder 317 lifts the front roller so that the film 319 is pushed into contact with the joined tube segments mounted on mandrel 302. The film 319 wipes off the excess bonding solution.

Although described in use for manufacturing stents, the apparatus 300 and method employed thereby can be used for different types of devices besides stents such as shunts, valves, filters and the like. Different types of therapeutic agents can be used in a single device by varying the amount and composition of the regions to which the agents are localized. Finally, tubes can be formed with agent dispersed in the polymer matrix, chemically attached to the polymer backbone, or coated onto the polymer.

The stent 100 according to the systems and methods of the present invention may be disease specific, and may be designed for local or regional therapy, or a combination thereof. They may be used to treat coronary and peripheral diseases such as vulnerable plaque, restenosis, bifurcated lesions, superficial femoral artery, below the knee, saphenous vein graft, arterial tree, small and tortuous vessels, and diffused lesions. The drugs or other agents delivered by the devices according to the present invention may be one or more drugs, bio-active agents such as growth factors or other agents, or combinations thereof The drugs or other agents of the device are ideally controllably released from the device, wherein the rate of release depends on either or both of the degradation rates of the bioabsorbable polymers comprising the device and the nature of the drugs or other agents. The rate of release can thus vary from minutes to years as desired.

Where the drug or agent is incorporated into the matrix of bioabsorbable polymers comprising the device, for example, the drug or agent will release by diffusion and during degradation of the device. The amount of drug or agent released by diffusion will tend to release for a longer period of time than occurs using coating techniques, and may often more effectively treat local and diffuse lesions or conditions thereof. For regional drug or agent delivery such diffusion release of the drugs or agents is effective as well. Polymer compositions and their diffusion and absorption characteristics will control drug elution profile for these devices. The drug release kinetics will be controlled by drug diffusion and polymer absorption. Initially, most of the drug will be released by diffusion from the device surfaces and bulk and will then gradually transition to drug release due to polymer absorption. There may be other factors that will also control drug release. If the polymer composition is from the same monomer units (e.g., lactide; glycolide), then the diffusion and absorption characteristics will be more uniform compared to polymers prepared from mixed monomers. Also, if there are layers of different polymers with different drug in each layer, then there will be more controlled release of drug from each layer. There is a possibility of drug present in the device until the polymer fully absorbs thus providing drug release throughout the device life cycle.

Bioabsorbable and/or biodegradable polymers consist of bulk and surface erodable materials. Surface erosion polymers are typically hydrophobic with water labile linkages. Hydrolysis tends to occur fast on the surface of such surface erosion polymers with no water penetration in bulk. The initial strength of such surface erosion polymers tends to be low however, and often such surface erosion polymers are not readily available commercially. Nevertheless, examples of surface erosion polymers include polyanhydrides such as poly (carboxyphenoxy hexane-sebacic acid), poly (fumaric acid-sebacic acid), poly (carboxyphenoxy hexane-sebacic acid), poly (imide-sebacic acid)(50-50), poly imide-carboxyphenoxy hexane) (33-67), and polyorthoesters (diketene acetal based polymers).

Bulk erosion polymers, on the other hand, are typically hydrophilic with water labile linkages. Hydrolysis of bulk erosion polymers tends to occur at more uniform rates across the polymer matrix of the device. Bulk erosion polymers exhibit superior initial strength and are readily available commercially. Examples of bulk erosion polymers include poly (α-hydroxy esters) such as poly (lactic acid), poly (glycolic acid), poly (caprolactone), poly (p-dioxanone), poly (trimethylene carbonate), poly (oxaesters), poly (oxaamides), and their co-polymers and blends. Some commercially readily available bulk erosion polymers and their commonly associated medical applications include poly (dioxanone) [PDS® suture available from Ethicon, Inc., Somerville, N.J.], poly (glycolide) [Dexon® sutures available from United States Surgical Corporation, North Haven, Conn.], poly (lactide)-PLLA [bone repair], poly (lactide/glycolide) [Vicryl® (10/90) and Panacryl® (95/5) sutures available from Ethicon, Inc., Somerville, N.J.], poly (glycolide/caprolactone (75/25) [Monocryl® sutures available from Ethicon, Inc., Somerville, N.J.], and poly (glycolide/trimethylene carbonate) [Maxon® sutures available from United States Surgical Corporation, North Haven, Conn.].

Other bulk erosion polymers are tyrosine derived poly amino acid [examples: poly (DTH carbonates), poly (arylates), and poly (imino-carbonates)], phosphorous containing polymers [examples: poly (phosphoesters) and poly (phosphazenes)], poly (ethylene glycol) [PEG] based block co-polymers [PEG-PLA, PEG-poly (propylene glycol), PEG-poly (butylene terephthalate)], poly (α-malic acid), poly (ester amide), and polyalkanoates [examples: poly (hydroxybutyrate (HB) and poly (hydroxyvalerate) (HV) co-polymers].

Of course, the stent 100 may be made from combinations of surface and bulk erosion polymers in order to achieve desired physical properties and to control the degradation mechanism. For example, two or more polymers may be blended in order to achieve desired physical properties and device degradation rate. Alternately, the device may be made from a bulk erosion polymer that is coated with a surface erosion polymer. The drug delivery device may be made from a bulk erosion polymer that is coated with a drug containing a surface erosion polymer. For example, the drug coating may be sufficiently thick that high drug loads may be achieved, and the bulk erosion polymer may be made sufficiently thick that the mechanical properties of the device are maintained even after all of the drug has been delivered and the surface eroded.

Shape memory polymers may also be used. Shape memory polymers are characterized as phase segregated linear block co-polymers having a hard segment and a soft segment. The hard segment is typically crystalline with a defined melting point, and the soft segment is typically amorphous with a defined glass transition temperature. The transition temperature of the soft segment is substantially less than the transition temperature of the hard segment in shape memory polymers. A shape in the shape memory polymer is memorized in the hard and soft segments of the shape memory polymer by heating and cooling techniques. Shape memory polymers may be biostable and bioabsorbable. Bioabsorbable shape memory polymers are relatively new and comprise thermoplastic and thermoset materials. Shape memory thermoset materials may include poly (caprolactone) dimethylacrylates, and shape memory thermoplastic materials may include poly (caprolactone) as the soft segment and poly (glycolide) as the hard segment.

In order to provide materials with high toughness, such as is often required for orthopedic implants, sutures, stents, grafts and other medical applications including drug delivery devices, the bioabsorbable polymeric materials may be modified to form composites or blends thereof. Such composites or blends may be achieved by changing either the chemical structure of the polymer backbone, or by creating composite structures by blending them with different polymers and plasticizers. The addition of plasticizers, which are generally low molecular weight materials, or a soft (lower glass transition temperature) miscible polymer, will depress the glass transition temperature of the matrix polymer system. In general, these additional materials that are used to modify the underlying bioabsorbable polymer should preferably be miscible with the main matrix polymer system to be effective.

Plasticizers that are added to the matrix of bioabsorbable polymer materials will make the device more flexible and typically reduces the processing temperatures in case of processing materials in melt. The plasticizers are added to the bioabsorbable materials of the device prior to or during processing thereof. As a result, degradation of drugs incorporated into the bioabsorbable materials having plasticizers added thereto during processing is further minimized.

Plasticizers or mixtures thereof suitable for use in the present invention may be selected from a variety of materials including organic plasticizers and those like water that do not contain organic compounds. Organic plasticizers include but not limited to, phthalate derivatives such as dimethyl, diethyl and dibutyl phthalate; polyethylene glycols with molecular weights preferably from about 200 to 6,000, glycerol, glycols such as polypropylene, propylene, polyethylene and ethylene glycol; citrate esters such as tributyl, triethyl, triacetyl, acetyl triethyl, and acetyl tributyl citrates, surfactants such as sodium dodecyl sulfate and polyoxymethylene (20) sorbitan and polyoxyethylene (20) sorbitan monooleate, organic solvents such as 1,4-dioxane, chloroform, ethanol and isopropyl alcohol and their mixtures with other solvents such as acetone and ethyl acetate, organic acids such as acetic acid and lactic acids and their alkyl esters, bulk sweeteners such as sorbitol, mannitol, xylitol and lycasin, fats/oils such as vegetable oil, seed oil and castor oil, acetylated monoglyceride, triacetin, sucrose esters, or mixtures thereof. Preferred organic plasticizers include citrate esters; polyethylene glycols and dioxane.

Polymer blends are commonly prepared to achieve the desired final polymer properties. In accordance with the present invention, polymer blends are prepared to increase the elongation at break values or ultimate strain and thereby improving the toughness of the material that will be used to prepare vascular devices such as stents. Selection of the materials is important in order to achieve high toughness values of the matrix polymer. Matching solubility parameters and increase in free volume is important for the polymer blends to achieve the desired performance. The main difference between adding a plasticizer and a polymer to the matrix polymer is the difference in their molecular weights. As mentioned earlier, plasticizers have lower molecular weight compared to a polymeric additive. However, some low molecular weight polymers may also be used as a plasticizer. It is possible to achieve high toughness values by adding low amounts of plasticizer compared to a polymeric additive. Relatively high molecular weight material has been used as the matrix material for the present invention. For example, the molecular weight (weight average) of PLGA resins may be above 300,000 Daltons. Thermodynamically, molecular weight plays a big role in miscibility of polymer systems. There is higher miscibility between polymer and a low molecular weight additive compared to a high molecular weight additive. As mentioned earlier, the addition of a miscible polymer will lower glass transition temperature, decrease modulus and tensile strength with an increase in the toughness values.

As an example of producing a composite or blended material, blending a stiff polymer such as poly (lactic acid), poly (glycolide) and poly (lactide-co-glycolide) copolymers with a soft and elastomeric polymer such as poly (caprolactone) and poly (dioxanone) tends to produce a material with high toughness and high stiffness. An elastomeric co-polymer may also be synthesized from a stiff polymer and a soft polymer in different ratios. For example, poly (glycolide) or poly (lactide) may be copolymerized with poly (caprolactone) or poly (dioxanone) to prepare poly (glycolide-co-caprolactone) or poly (glycolide-co-dioxanone) and poly (lactide-co-caprolactone) or poly (lactide-co-dioxanoe) copolymers. These elastomeric copolymers may then be blended with stiff materials such as poly (lactide), poly (glycolide) and poly (lactide-co-glycolide) copolymers to produce a material with high toughness and ductility. Alternatively, terpolymers may also be prepared from different monomers to achieve desired properties. For example, poly (caprolactone-co-glycolide-co-lactide) may be prepared in different ratios.

In addition to increasing the toughness values with the addition of the soft polymers, the absorption time may also be modified. For example, the blend of PLGA with polycaprolactone will increase the total absorption time of the blended material as polycaprolactone degrades slower than PLGA. The total absorption may be reduced for PLGA by blending it with faster degrading materials such as poly (dioxanone) and their copolymers with poly (glycolide) and poly (lactide); and copolymers of poly (glycolide) such as poly (caprolactone-co-glycolide). Reinforced composites may also be prepared by blending high modulus PGA fibers or bioabsorbable particulate fillers with PLGA to form composites in the presence of the plasticizers or soft materials to improve the modulus of the final material.

Melt blends of polymers, with melting points lower than the melting point of the bioabsorbable materials in which the drugs or other bio-active agents are to be incorporated, may also be added to the bioabsorbable materials that are to comprise the device. Adding the blends of polymers having the lower melting points also helps to reduce processing temperatures and minimize degradation of the drugs or agents thereby.

As described above, radiopaque materials may be added to the polymer blend from which the device is constructed to ensure visualization of the device as it is implanted in the patient. The radiopaque materials may be added directly to the matrix of bioabsorbable materials comprising the device during processing thereof resulting in fairly uniform incorporation of the radiopaque materials throughout the device. Alternately, the radiopaque materials may be added to the device in the form of a layer, a coating, a mark or band or powder at designated portions of the device depending on the geometry of the device and the process used to form the device. Coatings may be applied to the device in a variety of processes known in the art such as, for example, chemical vapor deposition (CVD), physical vapor deposition (PVD), electroplating, high-vacuum deposition process, microfusion, spray coating, dip coating, electrostatic coating, or other surface coating or modification techniques. Such coatings sometimes have less negative impact on the physical characteristics (eg., size, weight, stiffness, flexibility) and performance of the device than do other techniques.

Preferably, the radiopaque material does not add significant stiffness to the device so that the device may readily traverse the anatomy within which it is deployed. The radiopaque material should be biocompatible with the tissue within which the device is deployed. Such biocompatibility minimizes the likelihood of undesirable tissue reactions with the device. Inert noble metals such as gold, platinum, iridium, palladium, and rhodium are well-recognized biocompatible radiopaque materials. Other radiopaque materials include barium sulfate (BaSO₄), bismuth subcarbonate [(BiO)₂CO₃] and bismuth oxide. Preferably, the radiopaque materials adhere well to the device such that peeling or delamination of the radiopaque material from the device is minimized, or ideally does not occur. Where the radiopaque materials are added to the device as metal bands, the metal bands may be crimped at designated sections of the device. Alternately, designated sections of the device may be coated with a radiopaque metal powder, whereas other portions of the device are free from the metal powder.

The local delivery of therapeutic agent/therapeutic agent combinations may be utilized to treat a wide variety of conditions utilizing any number of medical devices, or to enhance the function and/or life of the device. For example, intraocular lenses, placed to restore vision after cataract surgery is often compromised by the formation of a secondary cataract. The latter is often a result of cellular overgrowth on the lens surface and can be potentially minimized by combining a drug or drugs with the device. Other medical devices which often fail due to tissue in-growth or accumulation of proteinaceous material in, on and around the device, such as shunts for hydrocephalus, dialysis grafts, colostomy bag attachment devices, ear drainage tubes, leads for pace makers and implantable defibrillators can also benefit from the device-drug combination approach.

Different drugs may be utilized as therapeutic agents, including sirolimus, heparin, everolimus, tacrolimus, paclitaxel, cladribine as well as classes of drugs such as statins. These drugs and/or agents may be hydrophilic, hydrophobic, lipophilic and/or lipophobic. The type of agent will play a role in determining the type of polymer. The amount of the drug in the coating may be varied depending on a number of factors including, the storage capacity of the coating, the drug, the concentration of the drug, the elution rate of the drug as well as a number of additional factors. The amount of drug may vary from substantially zero percent to substantially one hundred percent. Typical ranges may be from about less than one percent to about forty percent or higher. Drug distribution in the coating may be varied. The one or more drugs may be distributed in a single layer, multiple layers, single layer with a diffusion barrier or any combination thereof.

Although the present invention has been described above with respect to particular preferred embodiments, it will be apparent to those skilled in the art that numerous modifications and variations can be made to these designs without departing from the spirit or essential attributes of the present invention. Accordingly, reference should be made to the appended claims, rather than to the foregoing specification, as indicating the scope of the invention. The descriptions provided are for illustrative purposes and are not intended to limit the invention nor are they intended in any way to restrict the scope, field of use or constitute any manifest words of exclusion.

What is claimed is:

1. An apparatus made by a method comprising the steps of:
forming at least one first polymeric structure;
forming at least one additional polymeric structure having at least one agent contained therein;
joining the at least one additional polymeric structure to the at least one first structure to form a third structure having a pre-determined length; and
forming the third structure into a lattice of interconnected elements.

2. The apparatus made by the method of claim 1 wherein the third structure is formed into a stent.

3. The apparatus made by the method of claim 1 wherein the at least one first polymeric structure contains at least one agent therein.

4. The apparatus made by the method of claim 3 wherein the at least one agent contained within the first polymeric structure is different than the at least one agent of the at least one additional polymeric structure.

5. The apparatus made by the method of claim 4 wherein the amount of at least one agent in the at least one first polymeric structure and the at least one additional polymeric structure is different.

6. The apparatus made by the method of claim 3 wherein the at least one agent contained within the first polymeric structure is the same as the at least one agent of the at least one additional polymeric structure.

7. The apparatus made by the method of claim 6 wherein the amount of at least one agent in the at least one first polymeric structure and the at least one additional polymeric structure is equal.

8. The apparatus made by the method of claim 1 wherein the at least one agent comprises a therapeutic drug.

9. The apparatus made by the method of claim 1 wherein the at least one agent comprises a radiopaque agent.

10. The apparatus made by the method of claim 1 wherein the at least one agent comprises a plurality of therapeutic drugs.

11. The apparatus made by the method of claim 1 wherein the at least one agent comprises a plurality of radiopaque agents.

12. The apparatus made by the method of claim 1 wherein the at least one agent comprises at least one therapeutic agent and at least one radiopaque agent.

13. The apparatus made by the method of claim 1 wherein the at least one first polymeric structure and at least one additional polymeric structure are constructed from a bioabsorbable polymer.

14. The apparatus made by the method of claim 1 wherein the at least one additional polymer structure has a first layer having a first amount of at least one agent contained therein and a plurality of additional layers each having a second amount of at least one agent contained therein.

15. The apparatus of claim 14 wherein the thickness of the first layer is substantially the same as the thickness of each of the plurality of additional layers.

16. The apparatus of claim 15 wherein the ratio of the amount of the at least one agent contained within the first layer to the volume of the first layer is substantially the same as the ratio of the amount of at least one agent contained in each of the plurality of additional layers to the volume of each of the plurality of additional layers.

17. The apparatus of claim 16 wherein the first layer comprises a surface layer having a thickness in the range of five to fifteen microns.

18. The apparatus of claim 15 wherein the ratio of the amount of the at least one agent contained within one of the plurality of additional layers to the amount of agent contained in all the layers is greater than 10 percent.
